SEQUENCE LISTING (4 sequences)

SEQ ID NO: 1 - DNA, 20 bp, Artificial Sequence
  source: /note=“Description of Artificial Sequence Synthetic oligonucleotide”
caggatacatttctacagct

SEQ ID NO: 2 - protein, 154 aa, Homo sapiens [sequence omitted]

SEQ ID NO: 3 - DNA, 981 bp, Homo sapiens
gtttggggccagagtgggcgaggcgcggaggtctggcctataaagtagtcgcggagacggggtgctggtttgcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaaaaaaaaaaaaaaaaa

SEQ ID NO: 4 - protein, 543 aa, Homo sapiens
MSSFSYEPYYSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEISFLKKVHEEEIAELQAQIQYAQISVEMDVTKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRTTKSEMARYLKEYQDLLNVKMALDIEIAAYRKLLEGEETRLSFTSVGSITSGYSQSSQVFGRSAYGGLQTSSYLMSTRSFPSYYTSHVQEEQIEVEETIEAAKAEEAKDEPPSEGEAEEEEKDKEEAEEEEAAEEEEAAKEESEEAKEEEEGGEGEEGEETKEAEEEEKKVEGAGEEQAAKKKD

(19) United States
(12) Patent Application Publication
(10) Pub. No.: US 2024/0182903 A1
(43) Pub. Date: Jun. 6, 2024

(54) TREATMENT OF AMYOTROPHIC LATERAL SCLEROSIS

(71) Applicant: Biogen MA Inc., Cambridge, MA (US)

(72) Inventors: Yingying Liu, Cambridge, MA (US); Peng Sun, Lexington, MA (US); Stephanie Melillo Fradette, Concord, MA (US); Danielle LeeZetta Graham, Concord, MA (US); Janice Chun Yee Wong, Somerville, MA (US)

(21) Appl. No.: 18/283,526

(22) PCT Filed: Mar. 30, 2022
(86) PCT No.: PCT/US2022/022485

§ 371 Date: Sep. 22, 2023

Related U.S. Application Data
(60) Provisional application No. 63/168,972, filed on Mar. 31, 2021.

(51) Int. Cl. C12N 15/113; A61P 25/28

(52) U.S. Cl. CPC C12N 15/1137; A61P 25/28; C12N 2310/11; C12N 2310/315; C12N 2310/321

(57) ABSTRACT

The disclosure provides the use of neurofilament light chain levels for selecting a subject with a mutation in the superoxide dismutase 1 (SOD 1) gene for treatment with a SOD 1-targeting antisense oligonucleotide or salt thereof. The disclosed methods can be used in the treatment amyotrophic lateral sclerosis, including clinically presymptomatic amyotrophic lateral sclerosis.

### CROSS-REFERENCE TO RELATED APPLICATIONS

This application claims priority to U.S. Provisional Application No. 63/168,972, filed Mar. 31, 2021. The content of the foregoing application is incorporated by reference herein in its entirety.

### SEQUENCE LISTING

The instant application contains a Sequence Listing which has been submitted electronically in ASCII format and is hereby incorporated by reference in its entirety. Said ASCII copy, created on Mar. 24, 2022, is named 13751-0344WO1_SL.txt and is 7,948 bytes in size.

### FIELD

This disclosure relates to biomarkers for and treatment of amyotrophic lateral sclerosis.

### BACKGROUND

The soluble superoxide dismutase 1 (SOD1) enzyme (also known as Cu/Zn superoxide dismutase) is one of the superoxide dismutases that provides defense against oxidative damage of biomolecules by catalyzing the dismutation of superoxide to hydrogen peroxide (H₂O₂) (Fridovich, Annu. Rev. Biochem., 64:97-112 (1995)). The superoxide anion (O²⁻) is a potentially harmful cellular by-product produced primarily by errors of oxidative phosphorylation in mitochondria (Turrens, J. Physiol., 552:335-344 (2003))

Amyotrophic Lateral Sclerosis (ALS, also known as Lou Gehrig's disease) is a devastating progressive neurodegenerative disease affecting as many as about 17,000 Americans at any given time (Mehta, P., et al. (2018). “Prevalence of Amyotrophic Lateral Sclerosis—United States, 2015.” MMWR Morb Mortal Wkly Rep 67(46): 1285-1289).) Approximately 2% of ALS cases result from mutations in the gene encoding SOD1 (Bunton-Stasyshyn RKA, et al. Neuroscientist. 2015; 21:519-29). Mutations in the SOD1 gene are usually associated with a dominantly-inherited form of ALS, a disorder characterized by a selective degeneration of upper and lower motor neurons (Rowland, N. Engl. J Med., 2001, 344:1688-1700 (2001)).

The toxicity of mutant SOD1 is believed to arise from an initial misfolding (gain of function) reducing nuclear protection from the active enzyme (loss of function in the nuclei), a process that may be involved in ALS pathogenesis (Sau, Hum. Mol. Genet., 16:1604-1618 (2007)). The progressive degeneration of the motor neurons in ALS eventually leads to their death. When the motor neurons die, the ability of the brain to initiate and control muscle movement is lost. With voluntary muscle action progressively affected, patients in the later stages of the disease may become totally paralyzed.

There remains an unmet need for effective therapies for treating ALS. It is therefore an object herein to provide methods for the treatment of the disease.

### SUMMARY

This disclosure relates, in part, to treatment of amyotrophic lateral sclerosis associated with a mutation in the SOD1 gene in subjects (e.g., adults) with clinical symptoms/signs and in presymptomatic subjects (e.g., adults) with biomarker evidence of disease (e.g., neurofilament light chain level of at least 44 pg/mL).

Provided herein, in some aspects, are a treatment of amyotrophic lateral sclerosis associated with a mutation in the SOD1 gene in a human subject having a neurofilament light chain level of at least 44 pg/ml, e.g., where the human subject is clinically presymptomatic of ALS.

In one aspect, the disclosure features a method of treating amyotrophic lateral sclerosis associated with a mutation in the SOD1 gene in a human subject in need thereof by administering to the human subject a pharmaceutical composition comprising a therapeutically effective amount of an antisense oligonucleotide according to the following formula:

mCes Aeo Ges Geo Aes Tds Ads mCds Ads Tds Tds Tds mCds Tds Ads mCeo Aes Geo mCes Te (nucleobase sequence of SEQ ID NO: 1), wherein,
A=an adenine,
mC=a 5-methylcytosine
G=a guanine,
T=a thymine,
e=a 2′-O-methoxyethylribose modified sugar,
d=a 2′-deoxyribose sugar,
s=a phosphorothioate internucleoside linkage, and
o=a phosphodiester internucleoside linkage;
or a pharmaceutically acceptable salt thereof,
wherein the human subject has a neurofilament light chain level of at least 44 pg/ml prior to initiation of the treatment.

In some embodiments, the human subject has undergone an increase in neurofilament light chain level of at least 10 pg/ml prior to initiation of the treatment.

In some embodiments, the neurofilament light chain level is a level in a biological sample from the human subject, e.g., a blood, serum, plasma, or cerebrospinal fluid sample. In some embodiments, the neurofilament light chain level is a plasma level, e.g., a plasma level of at least 44 pg/ml and/or a plasma level increase of at least 10 pg/ml. In some embodiments, the neurofilament light chain level is a blood, serum, or cerebrospinal fluid level equivalent to the corresponding plasma level (e.g., equivalent to a plasma level of at least 44 pg/ml or plasma level increase of at least 10 pg/ml).

In some embodiments, the human subject has a neurofilament light chain level of at least 44 pg/ml prior to initiation of the treatment, and wherein the human subject has undergone an increase in neurofilament light chain level of at least 10 pg/ml prior to initiation of the treatment.

In some embodiments, the human subject has a plasma neurofilament light chain level of at least 44 pg/ml prior to initiation of the treatment, and wherein the human subject has undergone an increase in plasma neurofilament light chain level of at least 10 pg/ml prior to initiation of the treatment.

In another aspect, the disclosure features a method of treating amyotrophic lateral sclerosis associated with a mutation in the SOD1 gene in a human subject in need thereof by:
measuring a neurofilament light chain level of at least 44 pg/ml in a biological sample obtained from the human subject before initiation of treatment; and
administering to the human subject a pharmaceutical composition comprising a therapeutically effective amount of an antisense oligonucleotide according to the following formula:

mCes Aeo Ges Geo Aes Tds Ads mCds Ads Tds Tds Tds mCds Tds Ads mCeo Aes Geo mCes Te (nucleobase sequence of SEQ ID NO:1), wherein,
A=an adenine,
mC=a 5-methylcytosine
G=a guanine,
T=a thymine,
e=a 2′-O-methoxyethylribose modified sugar,
d=a 2′-deoxyribose sugar,
s=a phosphorothioate internucleoside linkage, and
o=a phosphodiester internucleoside linkage;
or a pharmaceutically acceptable salt thereof.

In some embodiments, the biological sample is blood, serum, plasma, or cerebrospinal fluid.

In some embodiments, the biological sample is blood.

In some embodiments, the biological sample is serum.

In some embodiments, the biological sample is plasma.

In some embodiments, the method further includes measuring in the human subject an increase in blood, serum, plasma, or cerebrospinal fluid neurofilament light chain level of at least 10 pg/ml prior to administering the antisense oligonucleotide or pharmaceutically acceptable salt thereof.

In some embodiments, the method further includes measuring in the human subject an increase in plasma neurofilament light chain level of at least 10 pg/ml prior to administering the antisense oligonucleotide or pharmaceutically acceptable salt thereof.

In some embodiments, the method further includes measuring in the human subject an increase in blood, serum, or cerebrospinal fluid level neurofilament light chain level equivalent to a plasma level increase of at least 10 pg/ml prior to administering the antisense oligonucleotide or pharmaceutically acceptable salt thereof.

In some embodiments of any of the methods described herein, the pharmaceutical composition is administered by intrathecal administration.

In some embodiments of any of the methods described herein, the pharmaceutical composition delivers a fixed dose of about 100 mg of the antisense oligonucleotide.

In some embodiments of any of the methods described herein, the mutation in the SOD1 gene is A4V.

In some embodiments of any of the methods described herein, the mutation in the SOD1 gene is A4V, H46R, G93S, A4T, G141X, D133A, V148G, N139K, G85R, G93A, V14G, C6S, 1113T, D49K, G37R, A89V, E100G, D90A, T137A, E100K, G41A, G41D, G41S, G13R, G72S, L8V, F20C, Q22L, H48R, T54R, 5591, V87A, T88deltaTAD, A89T, V97M, S105deltaSL, V118L, D124G, L114F, D90A, G12R, G147R, C6F, C6G, D101G, D101H, G114A, G85S, H43R, L106F, L106V, L38V, or R115G.

In some embodiments of any of the methods described herein, the human subject is clinically presymptomatic of amyotrophic lateral sclerosis.

In some embodiments of any of the methods described herein, the human subject is administered loading doses of the pharmaceutical composition followed by maintenance doses of the pharmaceutical composition.

In some embodiments where the human subject is administered loading doses of the pharmaceutical composition followed by maintenance doses of the pharmaceutical composition, the human subject is administered three loading doses, wherein the loading doses are administered 14 days apart.

In some embodiments where the human subject is administered loading doses of the pharmaceutical composition followed by maintenance doses of the pharmaceutical composition, the maintenance doses are administered every 28 days beginning 28 days after the third loading dose.

In some embodiments where the human subject is administered loading doses of the pharmaceutical composition followed by maintenance doses of the pharmaceutical composition, the loading doses and maintenance doses of the pharmaceutical composition are administered to the human subject as follows:
(i) a first loading dose of the pharmaceutical composition;
(ii) a second loading dose of the pharmaceutical composition administered 14 days after the first loading dose;
(iii) a third loading dose of the pharmaceutical composition administered 28 days after the first loading dose; and
(iv) a first maintenance dose of the pharmaceutical composition administered 28 days or 1 month after the third loading dose.

In some embodiments where the human subject is administered loading doses of the pharmaceutical composition followed by maintenance doses of the pharmaceutical composition, the loading doses and maintenance doses of the pharmaceutical composition are administered to the human subject as follows:
(i) a first loading dose in an amount sufficient to deliver a fixed dose of about 100 mg of the antisense oligonucleotide;
(ii) a second loading dose in an amount sufficient to deliver a fixed dose of about 100 mg of the antisense oligonucleotide, wherein the second loading dose is administered 14 days after the first loading dose;
(iii) a third loading dose in an amount sufficient to deliver a fixed dose of about 100 mg of the antisense oligonucleotide, wherein the third loading dose is administered 28 days after the first loading dose; and
(iv) a first maintenance dose in an amount sufficient to deliver a fixed dose of about 100 mg of the antisense oligonucleotide, wherein the first maintenance dose is administered 28 days after the third loading dose.

In accordance with any of the methods described herein, in some embodiments, the human subject is an adult, e.g., the human subject is at least 18 years of age, e.g., at least 18, 20, 25, 30, 35, 40, 45, 50, 55, 60, 65 years of age or older.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1 is a graph depicting a model-based estimate of geometric mean NfL level for participants with clinically manifested ALS 30 years or older at baseline with fast progressing mutations.

### DETAILED DESCRIPTION

This disclosure features the use of an antisense oligonucleotide, or salt thereof, for the treatment of amyotrophic lateral sclerosis associated with a mutation in the SOD1 gene in subjects (e.g., adults) with clinical symptoms/signs and in presymptomatic subjects (e.g., adults) with biomarker evidence of disease (e.g., neurofilament light chain level of at least 44 pg/ml).

### Amyotrophic Lateral Sclerosis

Amyotrophic lateral sclerosis (ALS) is a rare neurodegenerative disease resulting in loss of motor neurons within the cortex, brainstem, and spinal cord. Patients suffer from the progressive loss of muscle mass, strength, and function in bulbar, respiratory, and voluntary muscles. Decline is inevitable, with death, typically from respiratory failure, occurring 2 to 5 years, on average, following diagnosis. Although the majority of patients suffer from sporadic ALS, a smaller fraction of patients, approximately 2%, have an inherited, or familial, form of ALS caused by a variety of mutations in superoxide dismutase 1 (SOD1). Over 180 SOD1 mutations have been reported to cause this form of ALS (referred to as SOD1 ALS) since its initial discovery in 1993. The Amyotrophic Lateral Sclerosis Online Genetics Database (ALSoD). Institute of Psychiatry, Psychology & Neuroscience. Published 2015; Rosen, Nature, 364(6435):362 (1993)). Disease progression for individual mutations is variable, with survival of less than 15 months with the most severe mutations.

Approved treatments for ALS are riluzole (Rilutek®) and edaravone (Radicava™). Riluzole provides a modest increase in survival (2 to 3 months) without demonstrable improvement in strength or disability. Edaravone lessens functional decline as measured by the Revised Amyotrophic Lateral Sclerosis Functional Rating Scale (ALSFRS-R). The effect of edaravone on survival is unknown. No SOD1-specific ALS treatments are available.

### Superoxide Dismutase 1

Superoxide dismutase [Cu—Zn] also known as superoxide dismutase 1 (SOD1) is an enzyme that in humans is encoded by the SOD1 gene, located on chromosome 21.

SOD1 is a 32 kDa homodimer that forms a β-barrel and contains an intramolecular disulfide bond and a binuclear Cu/Zn site in each subunit. This Cu/Zn site holds the copper and a zinc ion and is responsible for catalyzing the disproportionation of superoxide to hydrogen peroxide and dioxygen.

SOD1 is one of three superoxide dismutases responsible for destroying free superoxide radicals in the body. The encoded isozyme is a soluble cytoplasmic and mitochondrial intermembrane space protein, acting as a homodimer to convert naturally occurring, but harmful, superoxide radicals to molecular oxygen and hydrogen peroxide. Hydrogen peroxide can then be broken down by another enzyme called catalase.

At least 180 mutations in the SOD1 gene have been linked to familial ALS (Conwit RA, J Neurol Sci., 251 (1-2):1-2 (2006); Al-Chalabi A, Leigh PN, Curr. Opin. in Neurol., 13(4):397-405 (2000); Redler RL, Dokholyan NV, Progress in Molecular Biology and Translational Science, 107:215-62 (2012)). However, wild-type SOD1, under conditions of cellular stress, has also been implicated in a significant fraction of sporadic ALS cases, which represent 90% of ALS patients. The most frequent mutations in human SOD1 are A4V in the United States; H46R in Japan; and G93S in Iceland. Other well-known human SOD1 mutations include: A4V, H46R, G93S, A4T, G141X, D133A, V148G, N139K, G85R, G93A, V14G, C6S, 1113T, D49K, G37R, A89V, E100G, D90A, T137A, E100K, G41A, G41D, G41S, G13R, G72S, L8V, F20C, Q22L, H48R, T54R, S591, V87A, T88deltaTAD, A89T, V97M, S105deltaSL, V118L, D124G, L114F, D90A, G12R, G147R, C6F, C6G, D101G, D101H, G114A, G85S, H43R, L106F, L106V, L38V, and R115G. Virtually all known ALS-causing SOD1 mutations act in a dominant fashion; a single mutant copy of the SOD1 gene is sufficient to cause the disease.

The amino acid sequence of human SOD1 can be found at UniProt P00441 and GENBANK Accession No. NP_000445, and is provided below:

(SEQ ID NO: 2)
MATKAVCVLK GDGPVQGIIN FEQKESNGPV KVWGSIKGLT
EGLHGFHVHE FGDNTAGCTS AGPHENPLSR KHGGPKDEER
HVGDLGNVTA DKDGVADVSI EDSVISLSGD HCIIGRTLVV
HEKADDLGKG GNEESTKTGN AGSRLACGVI
GIAQ

The nucleotide sequence encoding human SOD1 is provided at GENBANK Accession No. NM_000454.4, and is also provided below (the region recognized by the antisense oligonucleotide of this disclosure is underlined):

```
(SEQ ID NO: 3)
    1 gtttggggcc agagtgggcg aggcgcggag
        gtctggccta taaagtagtc gcggagacgg
  61 ggtgctggtt tgcgtcgtag tctcctgcag
        cgtctggggt ttccgttgca gtcctcggaa
121 ccaggacctc ggcgtggcct agcgagttat
        ggcgacgaag gccgtgtgcg tgctgaaggg
181 cgacggccca gtgcagggca tcatcaattt
        cgagcagaag gaaagtaatg gaccagtgaa
241 ggtgtgggga agcattaaag gactgactga
        aggcctgcat ggattccatg ttcatgagtt
301 tggagataat acagcaggct gtaccagtgc
        aggtectcac tttaatcctc tatccagaaa
361 acacggtggg ccaaaggatg aagagaggca
        tgttggagac ttgggcaatg tgactgctga
421 caaagatggt gtggccgatg tgtctattga
        agattctgtg atctcactct caggagacca
481 ttgcatcatt ggccgcacac tggtggtcca
        tgaaaaagca gatgacttgg gcaaaggtgg
541 aaatgaagaa agtacaaaga caggaaacgc
        tggaagtcgt ttggcttgtg gtgtaattgg
601 gatcgcccaa taaacattcc cttggatgta
        gtctgaggcc ccttaactca tctgttatcc
661 tgctagctgt agaaatgtat cctgataaac
        attaaacact gtaatcttaa aagtgtaatt
721 gtgtgacttt ttcagagttg ctttaaagta
        cctgtagtga gaaactgatt tatgatcact
781 tggaagattt gtatagtttt ataaaactca
        gttaaaatgt ctgtttcaat gacctgtatt
841 ttgccagact taaatcacag atgggtatta
        aacttgtcag aatttctttg tcattcaagc
901 ctgtgaataa aaaccctgta tggcacttat
        tatgaggcta ttaaaagaat ccaaattcaa
961 actaaaaaaa aaaaaaaaaa a
```

### SOD1 Antisense Oligonucleotide

“Antisense A” is a 5-10-5 MOE gapmer, having the sequence of (from 5′ to 3′) CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

Antisense A is described by the following chemical notation: mCes Aeo Ges Geo Aes Tds Ads mCds Ads Tds Tds Tds mCds Tds Ads mCeo Aes Geo mCes Te (SEQ ID NO: 1); wherein,
A=an adenine,
mC=a 5-methylcytosine
G=a guanine,
T=a thymine,
e=a 2′-O-methoxyethylribose modified sugar,
d=a 2′-deoxyribose sugar,
s=a phosphorothioate internucleoside linkage, and
o=a phosphodiester internucleoside linkage.

“2′-O-methoxyethyl” (also 2′-MOE and 2′-OCH₂CH₂—OCH₃ and MOE) refers to an O-methoxy-ethyl modification of the 2′ position of a furanose ring. A 2′-O-methoxyethyl modified sugar is a modified sugar.

“5-methylcytosine” means a cytosine modified with a methyl group attached to the 5′ position. A 5-methylcytosine is a modified nucleobase.

“Phosphorothioate linkage” or “phosphorothioate internucleoside linkage” means a linkage between nucleosides where the phosphodiester bond is modified by replacing one of the non-bridging oxygen atoms with a sulfur atom. A phosphorothioate linkage is a modified internucleoside linkage.

The Antisense A sequence can also be written in shorthand as follows:

(SEQ ID NO: 1)
5′_MeCA_{P-O}GG_{P-O}ATA^{Me}CATTT^{Me}CTA^{Me}C_{P-O}AG_{P-O}^{Me}C^{Me} U-3′

The underlined residues are 2′-MOE nucleosides. The P═O annotation reflects the location of phosphate diester linkages.

“2′-MOE nucleoside” (also 2′-O-methoxyethyl nucleoside) means a nucleoside comprising a MOE modified sugar moiety.

Antisense A is depicted by the following chemical structure:

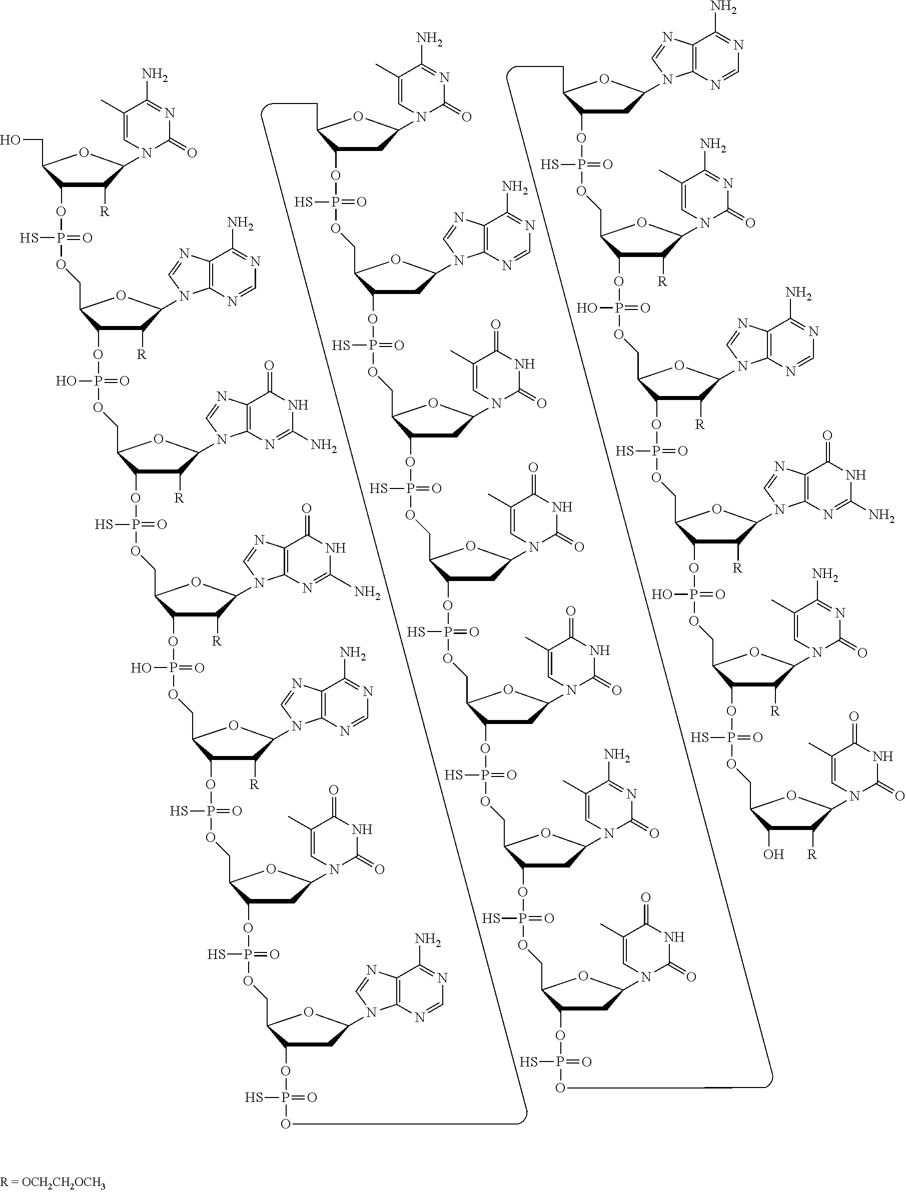

Antisense A is described in detail in U.S. Pat. No. 10,385,341, the content of which is incorporated herein by reference.

It is to be understood that in solution (e.g., in solution in a pharmaceutical composition) the antisense oligonucleotide may exist in free acid form, in a salt form, or a mixture thereof.

### Conjugated Antisense Oligonucleotides

Antisense oligonucleotides of this disclosure may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes. Antisense oligonucleotides can also be modified to have one or more stabilizing groups that are generally attached to one or both termini of antisense oligonucleotides to enhance properties such as, for example, nuclease stability. Included in stabilizing groups are cap structures. These terminal modifications protect the antisense oligonucleotide having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′stabilizing groups that can be used to cap one or both ends of an antisense oligonucleotide to impart nuclease stability include those disclosed in WO 03/004602.

### Compositions and Methods for Formulating Pharmaceutical Compositions

Antisense oligonucleotides or salts thereof of this disclosure may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

An antisense oligonucleotide, or salt thereof, targeted to a SOD1 nucleic acid can be used in pharmaceutical compositions by combining the antisense oligonucleotide, or salt thereof, with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense oligonucleotide, or salt thereof, targeted to a SOD1 nucleic acid and a pharmaceutically acceptable diluent.

An antisense oligonucleotide, or salt thereof, described herein may be formulated as a pharmaceutical composition for intrathecal administration to a subject.

Pharmaceutical compositions comprising antisense oligonucleotides of this disclosure encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense oligonucleotides and other bioequivalents. Suitable pharmaceutically acceptable salts include, but are not limited to, sodium and potassium salts.

### Methods of Treatment

The disclosure features methods of treating amyotrophic lateral sclerosis (e.g., clinically presymptomatic amyotrophic lateral sclerosis) associated with a mutation in the human SOD1 gene in a human subject in need thereof. The method involves administering to the human subject (e.g., by intrathecal administration) an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine. In certain instances, the antisense oligonucleotide is administered in a fixed dose of about 100 mg or 100 mg.

“About” in the context of the amount of a substance means+/−10% of the indicated value. “About” 100 mg of an antisense oligonucleotide includes 90 mg to 110 mg of the antisense oligonucleotide. In the context of temporal units, e.g., about 10 days or about 1 week, “about” means+/−3 days.

“Intrathecal or IT” means administration into the cerebrospinal fluid under the arachnoid membrane which covers the brain and spinal cord.

Also provided are methods of reducing human SOD1 protein synthesis in a human subject having a mutation in the human SOD1 gene associated with amyotrophic lateral sclerosis. The method involves administering to the human subject (e.g., by intrathecal administration) an antisense oligonucleotide, wherein the nucleobase sequence of the antisense oligonucleotide consists of CAGGATACATTTCTACAGCT (SEQ ID NO: 1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine. In certain instances, the antisense oligonucleotide is administered in a fixed dose of about 100 mg or 100 mg.

Also provided are methods of reducing human SOD1 mRNA levels in a human subject having a mutation in the human SOD1 gene associated with amyotrophic lateral sclerosis. The method involves administering to the human subject (e.g., by intrathecal administration) an antisense oligonucleotide, wherein the nucleobase sequence of the antisense oligonucleotide consists of CAGGATACATTTCTACAGCT (SEQ ID NO: 1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine. In certain instances, the antisense oligonucleotide is administered in a fixed dose of about 100 mg or 100 mg.

Also provided are methods of treating amyotrophic lateral sclerosis (e.g., clinically presymptomatic amyotrophic lateral sclerosis) associated with a mutation in the SOD1 gene in a human subject in need thereof, wherein the method entails administering to the human subject (e.g., by intrathecal administration) a pharmaceutical composition comprising an antisense oligonucleotide or a salt thereof, wherein the antisense oligonucleotide has the following structure:

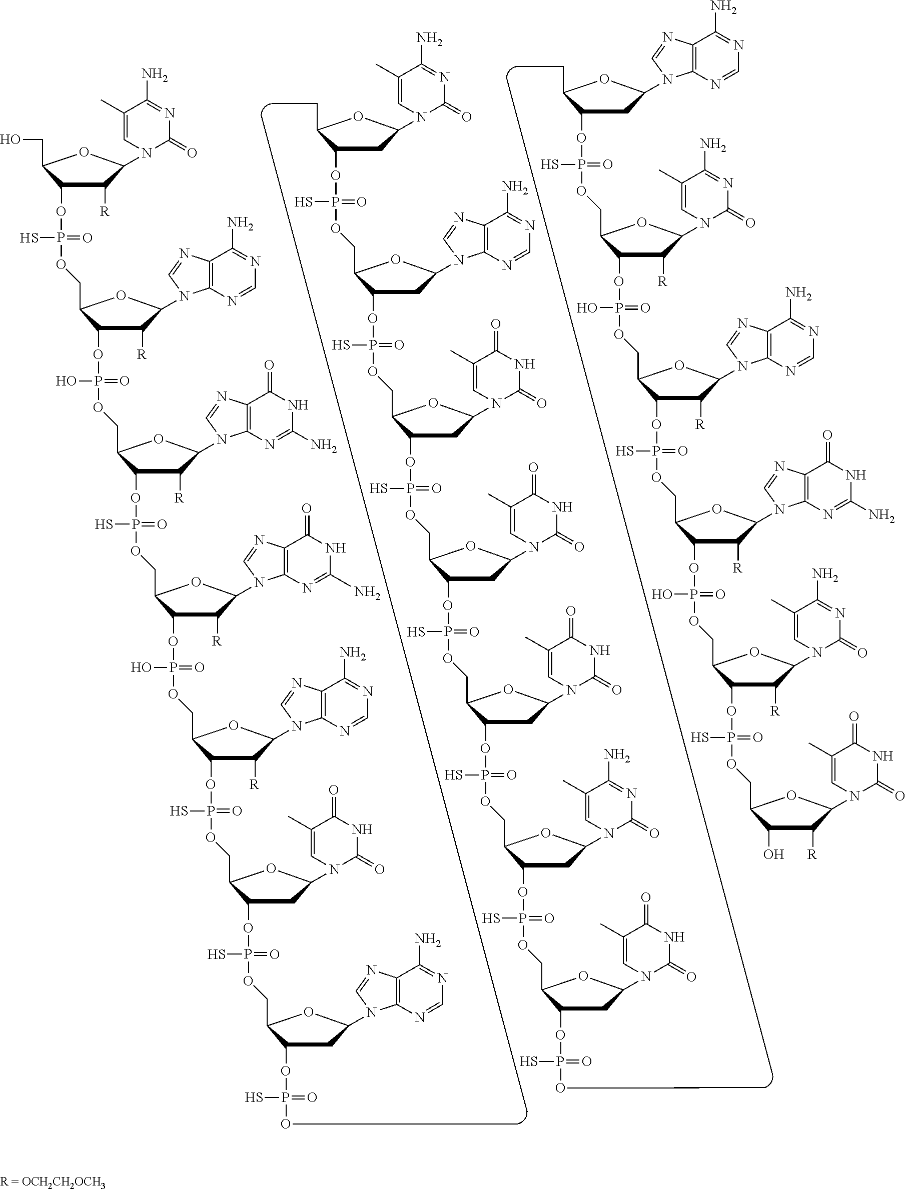

In certain instances, the antisense oligonucleotide or the salt thereof is administered at a dose equivalent to about 100 mg or 100 mg of the antisense oligonucleotide.

Also provided are methods of reducing human SOD1 protein synthesis in a human subject having a mutation in the human SOD1 gene associated with amyotrophic lateral sclerosis, wherein the method entails administering to the human subject (e.g., by intrathecal administration) a pharmaceutical composition comprising an antisense oligonucleotide or a salt thereof, wherein the antisense oligonucleotide has the following structure:

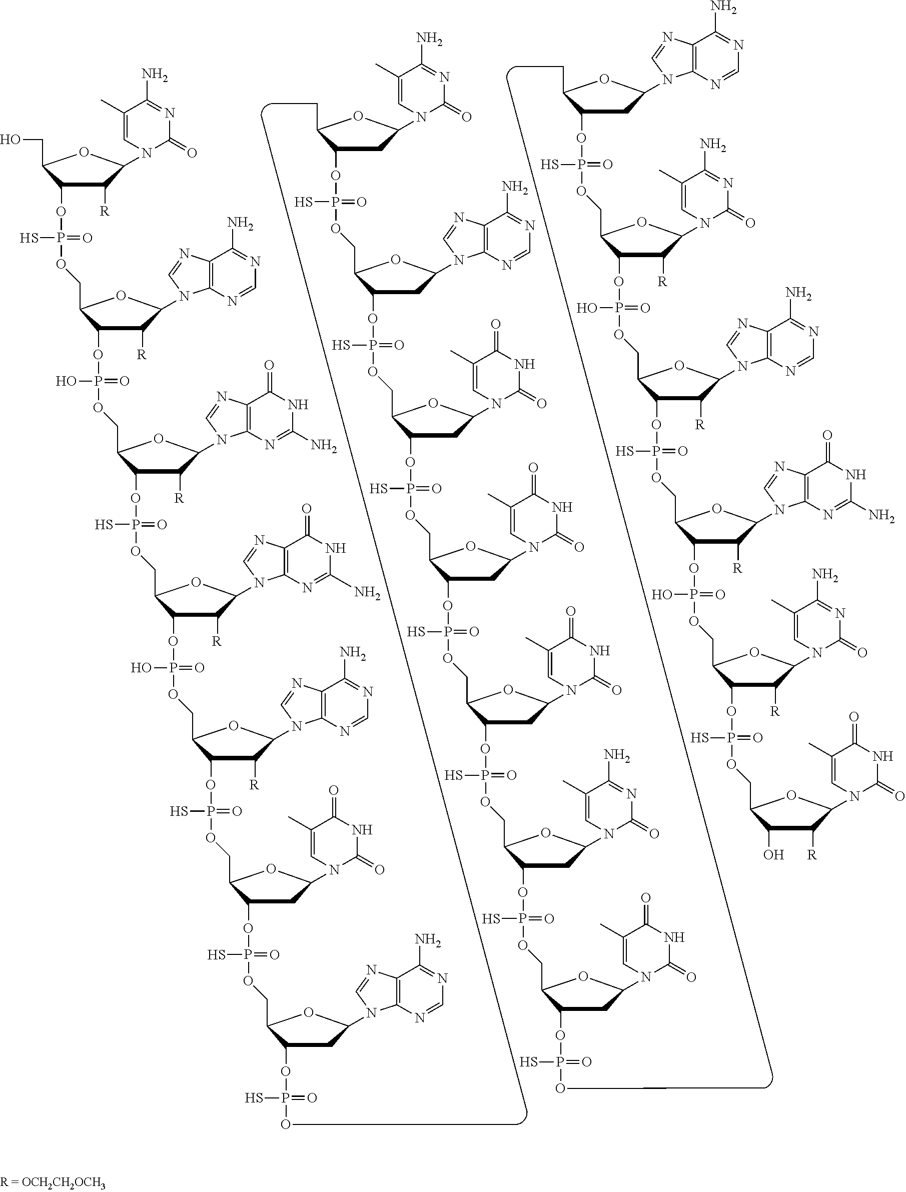

In certain instances, the antisense oligonucleotide or the salt thereof is administered at a dose equivalent to about 100 mg or 100 mg of the antisense oligonucleotide.

Also provided are methods of reducing human SOD1 mRNA levels in a human subject having a mutation in the human SOD1 gene associated with amyotrophic lateral sclerosis, wherein the method entails administering to the human subject (e.g., by intrathecal administration) a pharmaceutical composition comprising an antisense oligonucleotide or a salt thereof, wherein the antisense oligonucleotide has the following structure:

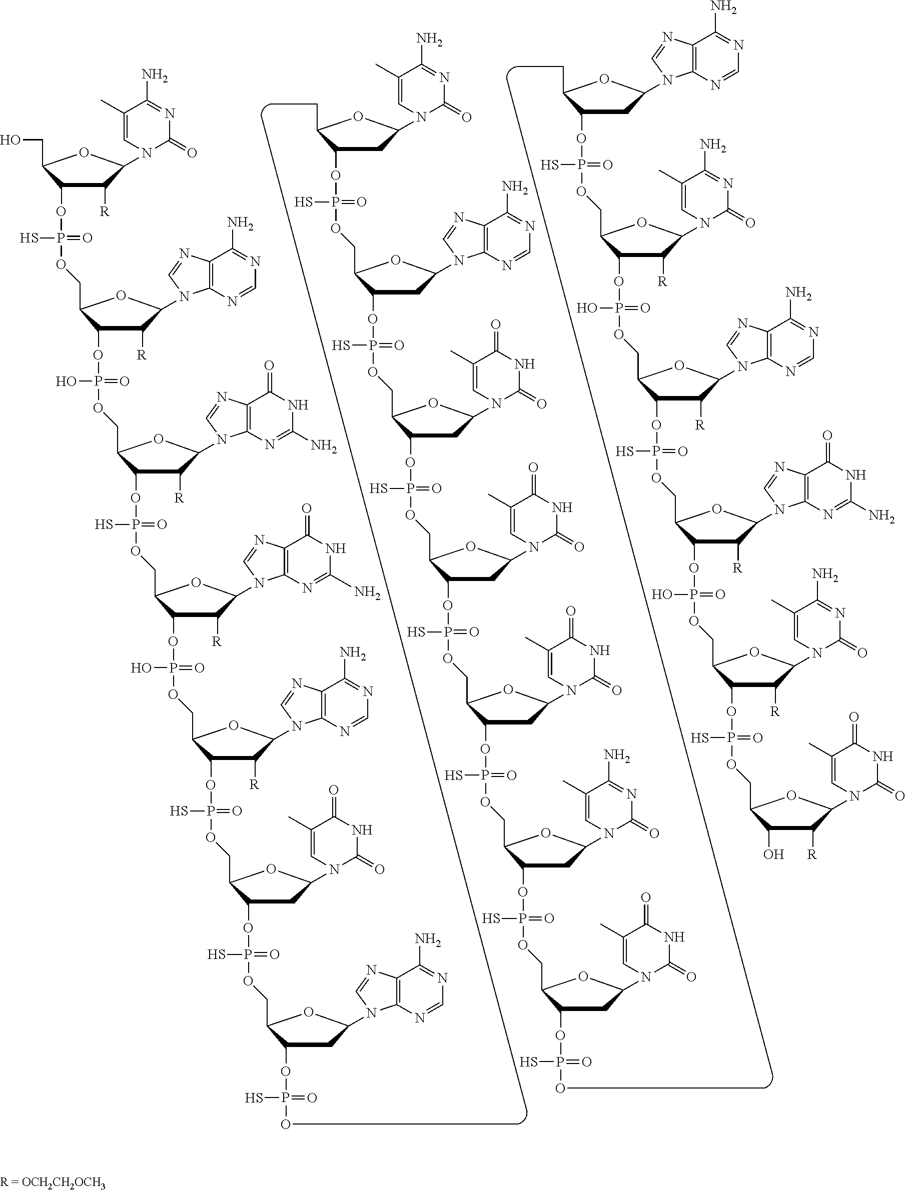

In certain instances, the antisense oligonucleotide or the salt thereof is administered at a dose equivalent to about 100 mg or 100 mg of the antisense oligonucleotide.

In some instances, an above-noted fixed dose of the antisense oligonucleotide, or salt thereof, is administered to the human subject once every week, once every two weeks, once every three weeks, or once every four weeks.

In some instances, the antisense oligonucleotide described herein is administered to the human subject as part of a pharmaceutical composition. In certain embodiments, the pharmaceutical composition is administered to the human subject in an amount sufficient to deliver a fixed dose of about 100 mg of the antisense oligonucleotide.

In certain embodiments, the antisense oligonucleotide, or salt thereof, is administered as a loading dose(s). In some embodiments, the antisense oligonucleotide is administered as a maintenance dose(s). In certain instances, the antisense oligonucleotide is administered as a loading dose(s) and followed by a maintenance dose(s). The loading dose(s) can be administered, e.g., every week, every two weeks, every three weeks, or every four weeks. The maintenance dose(s) can be administered, e.g., every week, every two weeks, every three weeks, or every four weeks after the last loading dose. In some instances, the maintenance dose(s) is administered every month.

“Loading Dose” means a dose administered during a dosing phase during which administration is initiated and steady state concentration of the drug (e.g., antisense oligonucleotide) achieved.

“Maintenance Dose” means a dose administered during a dosing phase after steady state concentration of the drug (e.g., antisense oligonucleotide) has been achieved.

In certain embodiments, the human subject is administered three loading doses of the antisense oligonucleotide, or salt thereof, followed by at least one (e.g., 1, 2, 3, 4, 5, 6, 7, 8, 9, 10, 11, 12, or more) maintenance dose. In some instances, the three loading doses are administered two weeks apart. In some instances, the three loading doses are administered 14 days apart. In some instances, the maintenance dose/doses are administered beginning 4 weeks after the third loading dose. In some instances, the maintenance dose/doses are administered every month beginning after the third loading dose. In some instances, the maintenance dose/doses are administered every 28 days beginning after the third loading dose.

The mutation in SOD1 may be any mutation in the human SOD1 gene that is associated with ALS. In some instances, the mutation is a slow-progressing ALS disease mutation. In other instances, the mutation is a fast-progressing ALS disease mutation. In certain instances, the mutation in the human SOD1 gene is one or more of A4V, H46R, G93S, A4T, G141X, D133A, V148G, N139K, G85R, G93A, V14G, C6S, 1113T, D49K, G37R, A89V, E100G, D90A, T137A, E100K, G41A, G41D, G41S, G13R, G72S, L8V, F20C, Q22L, H48R, T54R, 5591, V87A, T88deltaTAD, A89T, V97M, S105deltaSL, V118L, D124G, L114F, D90A, G12R, G147R, C6F, C6G, D101G, D101H, G114A, G85S, H43R, L106F, L106V, L38V, or R115G. In one particular embodiment, the human subject has an A4V mutation in the human SOD1 gene. In another particular embodiment, the human subject has a L106V mutation in the human SOD1 gene. In another particular embodiment, the human subject has an H46R mutation in the human SOD1 gene. In yet another particular embodiment, the human subject has a G93S mutation in the human SOD1 gene.

In certain instances, the mutation in the SOD1 gene is identified by a genetic test. Accordingly, identification of a subject suffering from or susceptible to ALS can be performed by genetic testing of the subject's SOD1 gene using assays known in the art, such as e.g., genetic sequencing.

Analysis of a subject's susceptibility to ALS can also be performed by analyzing the family history of the subject for ALS. Analysis of the family history may include a three-generation pedigree documenting ALS, a review of medical records and autopsy studies of family members, and identification of an autosomal dominant pattern of SOD1 mutation.

In certain embodiments, administration of a therapeutically effective amount of an antisense oligonucleotide, or a salt thereof, to a human subject is accompanied by monitoring of SOD1 levels in the human subject, to determine the human subject's response to administration of the antisense oligonucleotide, or salt thereof. A human subject's response to administration of the antisense oligonucleotide, or a salt thereof, may be used by a physician to determine the amount and duration of therapeutic intervention. In certain embodiments, the human SOD1 levels are monitored in CSF. In certain embodiments, the human SOD1 levels are monitored in plasma. In certain embodiments, the human SOD1 levels are monitored in blood. In certain embodiments, the human SOD1 levels are monitored in serum.

In certain embodiments, administration of an antisense oligonucleotide, or a salt thereof, results in reduction of SOD1 protein expression. In certain embodiments, administration of an antisense oligonucleotide, or a salt thereof, results in reduction of SOD1 protein expression by at least 10, 15, 20, 25, 30, 35, 40, 45, 50, 55, 60, 65, 70, 75, 80, 85, 90, 95 or 99%, or a range defined by any two of these values. In certain embodiments, the reduction of SOD1 protein expression is a reduction in the CSF. In certain embodiments, the reduction of SOD1 protein expression is a reduction in the plasma. In certain embodiments, the reduction of SOD1 protein expression is a reduction in blood. In certain embodiments, the reduction of SOD1 protein expression is a reduction in serum.

In certain embodiments, administration of an antisense oligonucleotide, or a salt thereof, results in improved motor function and respiration in the human subject. In certain embodiments, administration of the antisense oligonucleotide, or salt thereof, improves motor function and respiration by at least 10, 15, 20, 25, 30, 35, 40, 45, 50, 55, 60, 65, 70, 75, 80, 85, 90, 95 or 99%, or a range defined by any two of these values.

In certain embodiments, pharmaceutical compositions comprising an antisense oligonucleotide, or a salt thereof, are used for the preparation of a medicament for treating a human subject suffering or susceptible to ALS (e.g., a human subject having a mutation in SOD1 associated with ALS).

### Neurofilaments

This disclosure illustrates the use of neurofilament light chain levels as a marker for selecting a subject having a mutation in the SOD1 gene for treatment with an antisense oligonucleotide or salt thereof described herein. In some instances, a subject is selected for treatment if the subject has a neurofilament light chain level at or above a predetermined threshold. In some instances, a subject is selected for treatment if the subject has undergone an increase in a neurofilament light chain level of a predetermined minimum amount. In some instances, a subject is selected for treatment if the subject has a neurofilament light chain level at or above a predetermined threshold and has undergone an increase in a neurofilament light chain level of a predetermined minimum amount.

Assays for measuring neurofilament light chain in serum have been described (see, e.g., Gaiottino et al., PLoS ONE 8: e75091, 2013; Kuhle et al., J. Neurol. Neurosurg. Psychiatry 86(3): 273-279, 2014). Neurofilament light chain (NfL) (e.g., plasma or serum NfL) concentrations can be measured, for example, using ready-to-use enzyme linked immunosorbent assay (ELISA) diluent (Mabtech AB, Nacka Strand, Sweden), an electrochemiluminescence (ECL) immunoassay described in Gaiottino et al., PLoS ONE 8: e75091, 2013, or a single molecule array (SIMOA) method described in Disanto et al., Ann. Neurol. 81(6): 857-870, 2017. The three assay methods have been compared in Kuhl et al., Clinical Chemistry and Laboratory Medicine 54 (10): 1655-1661, 2016. The SIMOA assay (the Simoa NF-light Advantage kit) is commercially available from Quanterix Corp. (Lexington, MA, USA). In some embodiments, NfL (e.g., plasma or serum NfL) concentrations are measured using the Siemens Healthineers (SHL; Erlangen, Germany) NfL assay. In some instances, total NfL (e.g., phosphorylated and non-phosphorylated) is measured. In some embodiments, phosphorylated NfL is measured, and in some embodiments, non-phosphorylated NfL is measured.

In some embodiments, a subject (e.g., a subject clinically presymptomatic of amyotrophic lateral sclerosis) having a mutation in the SOD1 gene is selected for treatment if the subject has a neurofilament light chain level at or above a predetermined minimum threshold (e.g., a neurofilament light chain level of at least 44 pg/ml, e.g., as determined using the Siemens Healthineers NfL assay or an equivalent NfL level measured using a different assay).

In some embodiments, a subject (e.g., a subject clinically presymptomatic of amyotrophic lateral sclerosis) having a mutation in the SOD1 gene is selected for treatment if the subject has undergone an increase in a neurofilament light chain level of a predetermined minimum amount (e.g., an increase in neurofilament light chain level of at least 10 pg/ml, e.g., as determined using the Siemens Healthineers NfL assay or an equivalent NfL level measured using a different assay).

In some embodiments, a subject (e.g., a subject clinically presymptomatic of amyotrophic lateral sclerosis) having a mutation in the SOD1 gene is selected for treatment if the subject has a neurofilament light chain level at or above a predetermined minimum threshold (e.g., a neurofilament light chain level of at least 44 pg/ml, e.g., as determined using the Siemens Healthineers NfL assay or an equivalent NfL level measured using a different assay) and has undergone an increase in a neurofilament light chain level of a predetermined minimum amount (e.g., an increase in neurofilament light chain level of at least 10 pg/ml, e.g., as determined using the Siemens Healthineers NfL assay or an equivalent NfL level measured using a different assay). In some embodiments, without wishing to be bound by theory, it is believed that a change (e.g., increase) of at least 10 pg/mL in plasma NfL level from baseline can robustly account for the potential effect of aging on NfL level.

The amino acid sequence of human NF-L is provided in SEQ ID NO:4 and in Julien et al., Biochimica et Biohysica Acta, 909:10-20 (1987), UniProtKB—P07196, NCBI Reference Sequence: NP_006149.2, and NCBI Reference Sequence: NG_008492.1.

SEQ ID NO: 4
MSSFSYEPYYSTSYKRRYVETPRVHISSVRSGYSTARSAYSSYSAP
VSSSLSVRRSYSSSSGSLMPSLENLDLSQVAAISNDLKSIRTQEK
AQLQDLNDRFASFIERVHELEQQNKVLEAELLVLRQKHSEPSRFR
ALYEQEIRDLRLAAEDATNEKQALQGEREGLEETLRNLQARYEEE
VLSREDAEGRLMEARKGADEAALARAELEKRIDSLMDEISFLKKV
HEEEIAELQAQIQYAQISVEMDVTKPDLSAALKDIRAQYEKLAAK
NMQNAEEWFKSRFTVLTESAAKNTDAVRAAKDEVSESRRLLKAKT
LEIEACRGMNEALEKQLQELEDKQNADISAMQDTINKLENELRTT
KSEMARYLKEYQDLLNVKMALDIEIAAYRKLLEGEETRLSFTSVG
SITSGYSQSSQVFGRSAYGGLQTSSYLMSTRSFPSYYTSHVQEEQ
IEVEETIEAAKAEEAKDEPPSEGEAEEEEKDKEEAEEEEAAEEEE
AAKEESEEAKEEEEGGEGEEGEETKEAEEEEKKVEGAGEEQAAKK
KD

### Biological Samples

Suitable biological samples for the methods described herein include any biological fluid, cell, tissue, or fraction thereof, which includes analyte biomolecules of interest such as NF protein or nucleic acid (e.g., RNA (mRNA)). A biological sample can be, for example, a specimen obtained from a human subject or can be derived from such a subject. For example, a sample can be a tissue section obtained by biopsy, archived biological fluid, or cells that are placed in or adapted to tissue culture. In some instances, a biological sample is a biological fluid such as blood, serum, plasma, or cerebrospinal fluid (CSF), or such a sample absorbed onto a substrate (e.g., glass, polymer, paper). A biological sample can be further fractionated, if desired, to a fraction containing particular cell types. For example, a blood sample can be fractionated into serum or into fractions containing particular types of blood cells such as red blood cells or white blood cells (leukocytes). If desired, a sample can be a combination of samples from a subject such as a combination of a tissue and fluid sample.

The biological samples can be obtained from a subject having a mutation in the SOD1 gene (e.g., a SOD1 mutation described herein). In certain embodiments, the subject is clinically presymptomatic of amyotrophic lateral sclerosis.

Any suitable methods for obtaining the biological samples can be employed, although exemplary methods include, e.g., phlebotomy, fine needle aspirate biopsy procedure. Samples can also be collected, e.g., by microdissection (e.g., laser capture microdissection (LCM) or laser microdissection (LMD)).

Methods for obtaining and/or storing samples that preserve the activity or integrity of molecules (e.g., nucleic acids or proteins) in the sample are well known to those skilled in the art. For example, a biological sample can be further contacted with one or more additional agents such as buffers and/or inhibitors, including one or more of nuclease, protease, and phosphatase inhibitors, which preserve or minimize changes in the molecules (e.g., nucleic acids or proteins) in the sample. Such inhibitors include, for example, chelators such as ethylenediamine tetraacetic acid (EDTA), ethylene glycol bis(P-aminoethyl ether) N,N,N1,N1-tetraacetic acid (EGTA), protease inhibitors such as phenylmethylsulfonyl fluoride (PMSF), aprotinin, leupeptin, antipain, and the like, and phosphatase inhibitors such as phosphate, sodium fluoride, vanadate, and the like. Suitable buffers and conditions for isolating molecules are well known to those skilled in the art and can be varied depending, for example, on the type of molecule in the sample to be characterized (see, e.g., Ausubel et al. Current Protocols in Molecular Biology (Supplement 47), John Wiley & Sons, New York (1999); Harlow and Lane, Antibodies: A Laboratory Manual (Cold Spring Harbor Laboratory Press (1988); Harlow and Lane, Using Antibodies: A Laboratory Manual, Cold Spring Harbor Press (1999); Tietz Textbook of Clinical Chemistry, 3rd ed. Burtis and Ashwood, eds. W.B. Saunders, Philadelphia, (1999)). A sample also can be processed to eliminate or minimize the presence of interfering substances. For example, a biological sample can be fractionated or purified to remove one or more materials that are not of interest. Methods of fractionating or purifying a biological sample include, but are not limited to, chromatographic methods such as liquid chromatography, ion-exchange chromatography, size-exclusion chromatography, or affinity chromatography. For use in the methods described herein, a sample can be in a variety of physical states. For example, a sample can be a liquid or solid, can be dissolved or suspended in a liquid, can be in an emulsion or gel, or can be absorbed onto a material.

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### EXAMPLES

### Example 1: Use of a Neurofilament Threshold for Selection of Patients for Treatment with a SOD1 Antisense Oligonucleotide

A Phase 3, randomized, placebo-controlled study of Antisense A (as described herein) is conducted with a longitudinal natural history run-in in clinically presymptomatic adult SOD1 mutation carriers. SOD1 mutations included in this study are associated with high or complete penetrance and rapid progression to disease. SOD1 mutation carriers are considered clinically presymptomatic of amyotrophic lateral sclerosis (ALS) if they do not have clinically manifested ALS. Clinically manifested ALS is defined as the emergence of clinical symptoms or signs, which may be supported by EMG findings, that definitively indicate the emergence of ALS.

A plasma neurofilament light chain (NfL) threshold of 44 pg/mL was selected to identify study participants considered at high risk for onset of definite clinical symptoms/signs of ALS within 12 months (after reaching the NfL threshold). The plasma NfL threshold of 44 pg/mL was determined based on simulations from a NF trajectory model using data from presymptomatic familial amyotrophic lateral sclerosis (Pre-fALS) samples and selected to minimize the false positive rate while allowing for adequate time for enrollment and intervention between NF elevation and clinical onset.

Given the frequency of blood sampling in Pre-fALS, there are gaps between NfL levels, which in many cases impede the ability to identify how soon NfL began to elevate before onset of definite clinical symptoms/signs of ALS. To overcome this, an Emax model was fitted to the natural log transformed NfL concentration data using the Bayesian method for participants with clinically manifested ALS in Pre-fALS who were 30-years or older at baseline with fast progressing mutations. Separately, a Bayesian Weibull model was fitted to the time to emergence of clinically manifested ALS data for participants in Pre-fALS who were 30-years or older at baseline with fast progressing mutations. The NfL trajectory model allowed for the projection of time course from NfL elevation to emergence of clinically manifested ALS. Posterior predictive simulations from the fitted models were utilized to evaluate the performance of different NfL thresholds. Plasma NfL was analyzed herein using the Siemens Healthineers (SHL) NfL assay.

A NfL threshold of 44 pg/mL was derived based on the following factors:
1. Low false positive rate (e.g., less than 5%).
2. Adequate expected time from NfL elevation to clinical onset (e.g., greater than or equal to 2-3 months) to account for NfL processing time, screening, and randomization into the study.
3. Clinical experience with a subject not showing ALS symptom onset 12 months after NfL level reaching a 40 pg/mL threshold (but not reaching a 44 pg/mL threshold).

For a given NfL threshold, the false positive rate was evaluated among presymptomatic carriers with a NfL level below the threshold at enrollment and at least 12 months of follow-up time. A participant was considered a false positive if the NfL level exceeded the threshold at a postbaseline visit and the participant remained clinically presymptomatic for at least 12 months thereafter. The false negative rate was evaluated among all participants with clinically manifested ALS. A participant was considered a false negative if clinically manifested ALS emerged before reaching the NfL threshold.

For the NfL threshold of 44 pg/mL, the false positive rate was 0/24 (0%) and the false negative was 1/12 (8.3%). A threshold of 40 pg/mL was considered but rejected because of a higher false positive rate ( 1/24; 4.1%), while having the same false negative rate as 44 pg/mL. By minimizing the false positive rate with the NfL threshold of 44 pg/mL, healthy subjects are not exposed to a therapy that they may not need or that may not have a clinical effect on them.

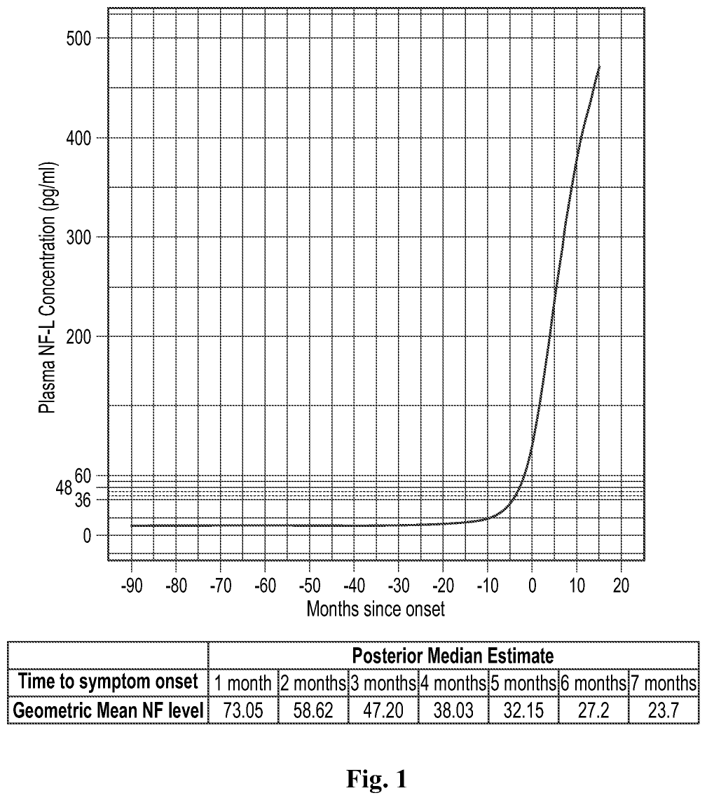

Based on the fitted NfL trajectory model, the geometric mean NfL levels prior to clinical symptom onset were estimated by month and displayed in FIG. 1. Three months prior to emergence of clinically manifested ALS, the expected geometric mean NfL level is approximately 44 pg/mL. For the NfL threshold of 44 pg/mL and a change from baseline in NfL of at least 10 pg/mL, posterior predictive probability of clinically manifested ALS for the placebo patients by 12 months is 77.66%.

The clinical study contains four parts, Part A, Part B, Part C, and Part D.

Part A is a natural history run-in, where participants do not receive study treatment, Antisense A or placebo. Participants in Part A are at least 18 years of age, e.g., at the time of informed consent. Participants in Part A have plasma NfL levels less than 44 pg/mL and no clinically manifested ALS during screening. Participants in Part A have one of the following SOD1 mutations confirmed during screening.
p.Ala5Thr (A4T, A5T)
p.Ala5Val (A4V, A5V)
p.Cys7Phe (C6F, C7F)
p.Cys7Gly (C6G, C7G)
p.Asp102Gly (D101G, D102G)
p.Asp102His (D101H, D102H)
p.Gly115Ala (G114A, G115A)
p.Gly42Ser (G41S, G42S)
p.Gly86Arg (G85R, G86R)
p.Gly86Ser (G85S, G86S)
p.Gly94Ala (G93A, G94A)
p.His44Arg (H43R, H44R)
p.Leu107Phe (L106F, L107F)
p.Leu107Val (L106V, L107V)
p.Leu39Val (L38V, L39V)
p.Arg116Gly (R115G, R116G)
p.Val149Gly (V148G, V149G)

Each of the parentheticals above refers to two alternate naming conventions for each amino acid substitution. As used elsewhere herein, each of the above amino acid substitutions is referred to by the variant identifier listed first in each parenthetical (e.g., A4T).

Alternatively, participants in Part A may have a SOD1 mutation other than one listed above that is adjudicated by an external Mutation Adjudication Committee for inclusion into the study. Adjudication must confirm that any additional SOD1 mutation included in the study has high or complete penetrance, and is associated with rapid disease progression.

Part B is a randomized, double-blind, placebo-controlled period in presymptomatic participants with plasma NfL levels greater than or equal to 44 pg/mL, a change from Part A baseline in NfL that is at least 10 pg/mL, and no alternative identifiable cause for the NfL elevation per the discretion of the investigator. Participants from Part A whose plasma NfL levels reach a level that is greater than or equal to 44 pg/mL and exhibit a change from baseline in NfL of at least 10 pg/mL and who have not developed clinically manifested ALS may be eligible to enroll in Part B. In Part B, participants are randomized in a 1:1 (Antisense A:placebo) ratio to receive one of the treatments administered by intrathecal injection (Antisense A 100 mg or placebo). Participants in Parts B receive 3 loading doses approximately every 14 days (i.e., Days 1, 15, and 29) and maintenance doses approximately every 28 days by intrathecal injection thereafter in a blinded manner.

The primary endpoint of the study is the proportion of participants with emergence of clinically manifested ALS within 12 months of initiating Part B. Secondary endpoints of the study include the proportion of participants with emergence of clinically manifested ALS within 24 months of initiating Part B, time to emergence of clinically manifested ALS, change in Revised Amyotrophic Lateral Sclerosis Functional Rating Scale (ALSFRS-R) total score, change in percent predicted slow vital capacity (SVC), ventilation assistance-free survival (VAFS; defined as time to the earliest occurrence of one of death or permanent ventilation), and overall survival.

Part C is an open-label extension study in which participants receive 100 mg of Antisense A by intrathecal injection. Participants from Part B who develop clinically manifested ALS are enrolled and monitored.

Part D is a randomized, double-blind, placebo-controlled period in participants from Part A with clinically manifested ALS. Participants are randomized in a 2:1 (Antisense A:placebo) ratio to receive via intrathecal injection either 100 mg Antisense A or placebo.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating amyotrophic lateral sclerosis associated with a mutation in the superoxide dismutase 1 (SOD1) gene in a human subject in need thereof, the method comprising administering to the human subject a pharmaceutical composition comprising a therapeutically effective amount of an antisense oligonucleotide according to the following formula:
mCes Aeo Ges Geo Aes Tds Ads mCds Ads Tds Tds Tds mCds Tds Ads mCeo Aes Geo mCes Te (nucleobase sequence of SEQ ID NO:1), wherein,
A=an adenine,
mC=a 5-methylcytosine
G=a guanine,
T=a thymine,
e=a 2′-O-methoxyethylribose modified sugar,
d=a 2′-deoxyribose sugar,
s=a phosphorothioate internucleoside linkage, and
o=a phosphodiester internucleoside linkage;
or a pharmaceutically acceptable salt thereof,
wherein the human subject has a neurofilament light chain level of at least 44 pg/ml prior to initiation of the treatment.

2. The method of claim 1, wherein the human subject has undergone an increase in neurofilament light chain level of at least 10 pg/ml prior to initiation of the treatment.

3. The method of claim 2, wherein the human subject has a blood, serum, or cerebrospinal fluid neurofilament light chain level equivalent to a plasma neurofilament light chain level of at least 44 pg/ml prior to initiation of the treatment, and wherein the human subject has undergone an increase in blood, serum, or cerebrospinal fluid neurofilament light chain level equivalent to an increase in plasma neurofilament light chain level of at least 10 pg/ml prior to initiation of the treatment.

4. The method of claim 2, wherein the human subject has a plasma neurofilament light chain level of at least 44 pg/ml prior to initiation of the treatment, and wherein the human subject has undergone an increase in plasma neurofilament light chain level of at least 10 pg/ml prior to initiation of the treatment.

5. A method of treating amyotrophic lateral sclerosis associated with a mutation in the SOD1 gene in a human subject in need thereof, the method comprising:
measuring a neurofilament light chain level of at least 44 pg/ml in a biological sample obtained from the human subject before initiation of treatment; and
administering to the human subject a pharmaceutical composition comprising a therapeutically effective amount of an antisense oligonucleotide according to the following formula:
mCes Aeo Ges Geo Aes Tds Ads mCds Ads Tds Tds Tds mCds Tds Ads mCeo Aes Geo mCes Te (nucleobase sequence of SEQ ID NO:1), wherein,
A=an adenine,
mC=a 5-methylcytosine
G=a guanine,
T=a thymine,
e=a 2′-O-methoxyethylribose modified sugar,
d=a 2′-deoxyribose sugar,
s=a phosphorothioate internucleoside linkage, and
o=a phosphodiester internucleoside linkage;
or a pharmaceutically acceptable salt thereof.

6. The method of claim 5, wherein the biological sample is blood, serum, plasma, or cerebrospinal fluid.

7. The method of claim 5, wherein the biological sample is plasma.

8. The method of claim 5 or 6, further comprising measuring in the human subject an increase in blood, serum, or cerebrospinal fluid level neurofilament light chain level equivalent to an increase in plasma neurofilament light chain level of at least 10 pg/ml prior to administering the antisense oligonucleotide or pharmaceutically acceptable salt thereof.

9. The method of any one of claims 5 to 7, further comprising measuring in the human subject an increase in plasma neurofilament light chain level of at least 10 pg/ml prior to administering the antisense oligonucleotide or pharmaceutically acceptable salt thereof.

10. The method of any one of the preceding claims, wherein the pharmaceutical composition is administered by intrathecal administration.

11. The method of any one of the preceding claims, wherein the pharmaceutical composition delivers a fixed dose of about 100 mg of the antisense oligonucleotide.

12. The method of any one of the preceding claims, wherein the mutation in the SOD1 gene is A4V.

13. The method of any one of the preceding claims, wherein the mutation in the SOD1 gene is A4V, H46R, G93S, A4T, G141X, D133A, V148G, N139K, G85R, G93A, V14G, C6S, 1113T, D49K, G37R, A89V, E100G, D90A, T137A, E100K, G41A, G41D, G41S, G13R, G72S, L8V, F20C, Q22L, H48R, T54R, 5591, V87A, T88deltaTAD, A89T, V97M, S105deltaSL, V118L, D124G, L114F, D90A, G12R, G147R, C6F, C6G, D101G, D101H, G114A, G85S, H43R, L106F, L106V, L38V, or R115G.

14. The method of any one of the preceding claims, wherein the human subject is clinically presymptomatic of amyotrophic lateral sclerosis.

15. The method of any one of the preceding claims, wherein the human subject is administered loading doses of the pharmaceutical composition followed by maintenance doses of the pharmaceutical composition.

16. The method of claim 15, wherein the human subject is administered three loading doses, and wherein the loading doses are administered 14 days apart.

17. The method of claim 16, wherein the maintenance doses are administered every 28 days beginning 28 days after the third loading dose.

18. The method of claim 15, wherein the loading doses and maintenance doses of the pharmaceutical composition are administered to the human subject as follows:
(i) a first loading dose of the pharmaceutical composition;
(ii) a second loading dose of the pharmaceutical composition administered 14 days after the first loading dose;
(iii) a third loading dose of the pharmaceutical composition administered 28 days after the first loading dose; and
(iv) a first maintenance dose of the pharmaceutical composition administered 28 days or 1 month after the third loading dose.

19. The method of claim 15, wherein the loading doses and maintenance doses of the pharmaceutical composition are administered to the human subject as follows:
(i) a first loading dose in an amount sufficient to deliver a fixed dose of about 100 mg of the antisense oligonucleotide;
(ii) a second loading dose in an amount sufficient to deliver a fixed dose of about 100 mg of the antisense oligonucleotide, wherein the second loading dose is administered 14 days after the first loading dose;
(iii) a third loading dose in an amount sufficient to deliver a fixed dose of about 100 mg of the antisense oligonucleotide, wherein the third loading dose is administered 28 days after the first loading dose; and
(iv) a first maintenance dose in an amount sufficient to deliver a fixed dose of about 100 mg of the antisense oligonucleotide, wherein the first maintenance dose is administered 28 days after the third loading dose.
